(19) United States
(12) Patent Application Publication
(10) Pub. No.: US 2017/0186111 A1
(43) Pub. Date: Jun. 29, 2017

(54) RESTAURANT OPERATION SYSTEM AND METHOD

(71) Applicant: Steven H. Leach, Toronto (CA)

(72) Inventor: Steven H. Leach, Toronto (CA)

(21) Appl. No.: 15/312,810

(22) PCT Filed: Jun. 5, 2015
(86) PCT No.: PCT/CA2015/050519

§ 371 Date: Nov. 21, 2016

Related U.S. Application Data
(60) Provisional application No. 62/007,997, filed on Jun. 5, 2014.

(51) Int. Cl. G06Q 50/12; G01N 33/497; G06K 9/00; G06Q 20/20

(52) U.S. Cl. CPC G06Q 50/12; G06Q 20/20; G01N 33/4972; G06K 9/00221; G06K 2009/00322

(57) ABSTRACT

The system is for restaurants, nightclub and bars of the type wherein a plurality of running tabs are maintained, each tab including details of the food and drink items delivered to a service point. The improvement includes a database and a computing functionality. The database includes: details of the amount of alcohol in each drink item; details of the approximate time of delivery of said each drink item to the service point; and details of the approximate patron body mass associated with the service point. The computing functionality is adapted to: carry out calculations to predict the maximum breath alcohol concentration that each patron at the service point may experience over time, using the approximate patron body mass associated with the service point and estimated elimination rates; and create a signal when the maximum breath alcohol exceeds a predetermined limit.

### FIELD

The invention relates to the field of restaurant operation.

### BACKGROUND

It is known for restaurants to install automation systems. In a known modern type of this system, patrons themselves order food and drink via a computer system. In a common type of this known modern type of system, a tablet-type computer is positioned at each seat, and provided with an ordering functionality. In some jurisdictions, the use of self-order systems poses complications in respect of the sale of alcoholic beverages, as it is commonplace for persons trained in the art of beverage service to be employed to monitor consumption and look for signs of impairment.

### SUMMARY OF THE INVENTION

Forming one aspect of the invention is an improved point-of-sale system for restaurants, nightclub and bars, of the type wherein a plurality of running tabs are maintained, each tab including details of the food and drink items delivered to a service point.

The improvement comprises a database and a computing functionality.

The database includes: details of the amount of alcohol in each drink item; details of the approximate time of delivery of said each drink item to the service point; and details of the approximate body mass associated with the service point.

The computing functionality is adapted to carry out calculations to predict maximum expected breath alcohol concentration associated with the service point, using the approximate body mass associated with the service point and the details of the amount of alcohol delivered to the service point and the times of said deliveries; and create a signal when the maximum expected breath alcohol exceeds a predetermined limit.

According to another aspect of the invention, the service point can be defined by a patron.

According to another aspect of the invention, the system can be of the type wherein order-taking is done by restaurant staff, and the signal notifies restaurant staff to take an action selected from the group of actions consisting of: cessation of alcohol sales to the patron for a period of time; visual observation of the patron prior to acceptance of further orders; visual observation of the patron prior to delivery of further orders; complete cessation of alcohol sales to the patron.

According to another aspect of the invention, the system can be of the type wherein each seat in the restaurant is provided with a computing device for acceptance of orders and the signal is sent to the device provided to the seat occupied by the patron.

According to another aspect of the invention, the system can further comprise a breath alcohol tester, and the signal can notify the patron that alcoholic beverage sales to the patron will be curtailed for a period of time associated with the maximum expected breath alcohol concentration, unless a breath sample is delivered to the tester by the patron that demonstrates that the prediction is false.

According to another aspect of the invention, the database can include details of the approximate age and sex of the patron associated with the service point and is populated by: manual entry by restaurant staff; or automation associated with optical imaging and face analysis software; and the elimination rate is approximated based upon the approximate age and sex of the patron.

According to another aspect of the invention, each computing device can have an optical imaging device and the database can be populated with the approximate age and sex of the patrons by automation associated with the optical imaging devices.

According to another aspect of the invention, a scale can be associated with the breath alcohol tester and the database can be updated based upon measurements collected from the scale for the purpose of future calculations.

According to another aspect of the invention, upon delivery of a breath sample to the breath alcohol tester by a patron, the computing functionality updates the elimination rate for the purpose of future calculations.

### DETAILED DESCRIPTION

The invention relates to improvements in point-of-sale systems for restaurants, nightclub and bars, the systems being of the type wherein a plurality of running tabs are maintained, each tab including details of the food and drink items delivered to a service point, typically a patron, or more accurately, the seat occupied by the patron.

In an exemplary embodiment, advantageously used in point-of-sale systems of the type wherein each seat in a restaurant is provided with a computing device for acceptance of orders, the improvement comprises: database improvements, computing functionality improvements, a breath alcohol tester; order entry device improvements; and seating improvements.

As part of the database improvements, the database includes:
details of the amount of alcohol in each drink item on the menu
details of the approximate time of delivery of said each drink item to the service point, i.e. seat
details of the patron associated with the service point, namely, approximate age, mass and sex
details of typical time of consumption of alcoholic drinks on the menu

The computing functionality improvements include the ability to carry out calculations to predict the maximum breath alcohol concentration each patron may experience over time, using the details of the alcoholic beverages delivered to the patron, the details in the database of the patron and assumed times of consumption of such beverages.

The computing functionality improvements include the ability to carry out calculations, in the context of a patron that has placed more than one alcoholic beverage order, to predict the maximum breath alcohol concentration said patron would experience over time, if the patron were to repeat, at the same frequency, any of the previous orders made by the patron.

The computing functionality improvement also includes the ability to create a signal if the maximum predicted breath alcohol concentration exceeds a predetermined limit.

The breath alcohol tester has an imaging device and a keypad but is otherwise of a conventional institution type, i.e. as found in many restaurants and nightclubs.

The order entry device improvements include the incorporation, in each computing device, of an optical imaging device, and age and sex estimation software.

The seating improvement includes a mass sensor associated with each seat in the venue and adapted to measure the mass of the occupant.

In use, the exemplary system generally enables patrons to interact with the restaurant in the manner that is conventional therein, namely, patrons each use their respective tablets to order food and drink.

However, in the background, a database is populated with the approximate age, sex and mass of each patron, by computer analysis of the facial images captured by the tablets and via the seat sensors.

Weight sensors in seats are well-known and as such, further detail is neither required nor provided.

Similarly, age and sex estimation software is conventional, and as such, further detail is neither required nor provided:

http://blog.laptopmag.com/new.face-com-tool-can-guess-your-age-determine-gender-and-mood-hands-on

Also in the background, as drink orders are taken by the system, the computing facility is constantly carrying out calculations, to predict

i. the maximum breath alcohol concentration each patron may experience over time, using the details of the alcoholic beverages delivered to the patrons, the details in the database of the patrons and assumed times of consumption of such beverages

ii. the maximum breath alcohol concentration said patron would experience over time, if the patron were to repeat, at the same frequency, any of the previous orders made by the patron.

Persons of ordinary skill readily appreciate that the calculation of breath alcohol, taking into account mass, sex, age, and alcohol consumed, is easily done using conventional statistic analysis techniques and based upon Ueno's formula, Widmark's formula or combinations thereof. Accordingly, further detail is neither provided nor required.

Persons of ordinary skill will also readily appreciate that consumption times of beverages can be readily obtained, and will vary for various factors, including music volume. For the purpose of the calculation, a global constant can be assumed, for example, 14.5 minute for an 8 oz beer.

[Alcoholism: Clinical & Experimental Research, Gueguen et al. (2008)]

Alternatively, each restaurant could, for example, keep and use internal statistics. This would be a matter of routine to persons of ordinary skill, and as such, further detail is neither required nor provided.

On receipt of an order which the calculations suggest will cause the patron to experience a breath alcohol concentration in excess of a predetermined threshold level, i.e. threshold condition (i) above, typically the legal limit, the system will notify the patron accordingly and reject the order. Thus, if a relatively low-mass person, having previously ordered, for example, a single two-ounce drink, were to relatively quickly thereafter attempt to order a further two-ounce drink, the system might reject that order, but accept a single-ounce drink order. In the event that an order is rejected, but the patron has previously ordered drinks that would be available for purchase, i.e. contains less alcohol, the system advises the patron accordingly.

It will be evident that this functionality has significant advantage, in that it helps to reduce overindulgence, with commensurate impacts upon public safety.

In the context of a calculation that exceeds threshold condition (ii) above, the system will advise the patron that, if drinking patterns continue, future sales may be rejected, unless a breath sample is delivered to the tester by the patron that demonstrates that the prediction is false. The system will also deliver to the patron a key word for the alcohol breath tester, if he or she wishes to use same, and provide an indication to the patron of the amount of time which must pass before the system will accept orders for drinks of the types which the patron has previously ordered.

It will be evident that this functionality has significant advantage as well, in that it allows patrons to avoid the embarrassment associated with a refusal-to-serve; the patron can continue to enjoy his or her evening with others, ordering low- or no-alcohol drinks as required to maintain predicted sobriety.

For those patrons who believe that the prediction made by the system is inaccurate, for whatever reason, delivery of a breath sample involves attendance by the patron at the alcohol breath tester, entry of the key word and delivery of a breath sample in a generally conventional manner.

However, in the background, the imaging device associated with the breath alcohol tester will image the person delivering the breath sample and compare the image captured by the device against the image captured by the tablet and use conventional image matching software to ensure that the person delivering the sample is, indeed, the patron to whom the keyword was given.

If the appropriate keyword has been entered, but the comparison does not indicate a match, i.e. if it appears that a patron has asked a third party to provide a breath sample, the system will notify the sample provider accordingly and advise that the breath sample could not be accepted.

Upon delivery of a breath sample to the breath alcohol tester by a patron, i.e. in circumstances wherein the image captured by the breath tester corresponds to the image of the patron to whom the keyword was given, the computing functionality updates the elimination rate for the purpose of future calculations, and, in the event that the breath sample demonstrates that the earlier calculation was sufficiently inaccurate to modify the service standards applicable to the patron, advises the patron accordingly. So as to safeguard against service to minors, and overindulgence by one patron facilitated by another, in the event of an override, a signal is sent to the restaurant management, to monitor the patron in question.

Whereas a single embodiment is herein described, it will be evident that variations are possible.

For example, whereas in the exemplary embodiment, the system is used with a self-order system, this is not strictly necessary. The system could be employed to some advantage in the context of traditional point-of-sale systems, wherein servers accept orders by memory or on paper and enter the orders at stations scattered throughout the restaurant, but in this case, the signal would merely be relayed to the server, who, when delivering the order, would more carefully examine the patron for signs of impairment. The system could also be employed in the context of mobile point-of-sale systems, wherein servers enter orders on mobile devices such as tablets.

The system could also be used in the context of self-serve type operations, such as the Beer Table or Beer Wall distributed by Ellickson USA, wherein customers are provided with magnetic fobs which actuate draft taps. The use of magnetic fobs would largely obviate the need for imaging devices.

Further, whereas in the exemplary embodiment, the age and sex of each patron is approximated by a computer software program and image recognition, these details could be entered into the system manually, by a server or host.

Additionally, whereas in the exemplary embodiment, the service location is defined by a seat and assumes each patron orders and consumes his or her own drinks, the system could be used to some advantage in the context of service locations defined by tables, and also in the context of patrons grouped at a table sharing drinks. In this context, the alcohol consumed by each legal age patron could be assumed to be an equal share of the total alcohol delivered to the table; in all other respects, the system could function equivalently.

Further, whereas in the exemplary embodiment, the mass of the patron is gathered by a seat sensor, this could be approximated and entered manually into the system by a server or host, or could be estimated by a computer software program and image recognition hardware.

A scale could optionally be provided, and integrated with the breath tester, so that an accurate mass of the patron could be gathered and used by the system for future calculations. This would be of significant advantage in the manual weight entry embodiments, but could also be of advantage even in establishments with seat sensors, in that it could permit lower precision sensors to be utilized in the seats.

As yet another example, the system could be embodied to function with individual breath testers and cell phones. For example, a restaurant could: allow patrons to register their phones and breath testers; send signals to the phones; and receive samples from the personal breath testers. This would be particularly beneficial in the context of self-serve ordering via smart phone, accommodated, for example, via indoor positioning systems such as that sold at http://www.indooratlas.com/

Accordingly, the invention should be understood as limited only by the accompanying claims, purposively construed.

1. An improved point-of-sale system for restaurants, nightclub and bars, of the type wherein a plurality of running tabs are maintained, each tab including details of the food and drink items delivered to a service point, the improvement comprising:
a database that includes: details of the amount of alcohol in each drink item; details of the approximate time of delivery of said each drink item to the service point; and details of the approximate patron body mass associated with the service point;
a computing functionality adapted to
carry out calculations to predict the maximum breath alcohol concentration that each patron at the service point may experience over time, using the approximate patron body mass associated with the service point and estimated elimination rates; and
create a signal when the maximum breath alcohol exceeds a predetermined limit.

2. A system according to claim 1, wherein the service point is defined by a patron.

3. A system according to claim 2, wherein the system is of the type wherein order-taking is done by restaurant staff, and the signal notifies restaurant staff to take an action selected from the group of actions consisting of: cessation of alcohol sales; visual observation of the patron prior to acceptance of further orders; visual observation of the patron prior to delivery of further orders.

4. A system according to claim 2, wherein the system is of the type wherein each seat in the restaurant is provided with a computing device for acceptance of orders and the signal is sent to the device provided to the seat occupied by the patron.

5. A system according to claim 4, further comprising a breath alcohol tester, and wherein the signal notifies the patron that alcoholic beverage sales to the patron will be curtailed for a period of time associated with the predicted maximum breath alcohol concentration, unless a breath sample is delivered to the tester by the patron that demonstrates that the prediction is false.

6. A system according to claim 5, wherein
the database includes details of the approximate age and sex of the patron associated with the service point and is populated by: manual entry by restaurant staff; or automation associated with optical imaging and face analysis software; and
the elimination rate is approximated based upon the approximate age and sex of the patron.

7. A system according to claim 6, wherein each computing device has an optical imaging device and the database is populated with the approximate age and sex of the patrons by automation associated with the optical imaging devices.

8. A system according to claim 5, further comprising a scale associated with the breath alcohol tester and the database is updated based upon measurements collected from the scale for the purpose of future calculations.

9. A system according to claim 5, wherein, upon delivery of a breath sample to the breath alcohol tester by a patron, the computing functionality updates the elimination rate for the purpose of future calculations.
